(19) United States
(12) Patent Application Publication
(10) Pub. No.: US 2025/0375663 A1
(43) Pub. Date: Dec. 11, 2025

(54) USER EFFORT MEASUREMENT DURING WORKOUT SESSION

(71) Applicant: Apple Inc., Cupertino, CA (US)

(72) Inventors: Julia K. Nichols, Alameda, CA (US); Di Wu, San Jose, CA (US); En-Tzu Yang, Cupertino, CA (US); Gregory Darnell, Palo Alto, CA (US); Hui Chen, Los Altos, CA (US); James P. Ochs, San Francisco, CA (US); Paige N. Stanley, San Jose, CA (US); Shruti S. Kulkarni, Sunnyvale, CA (US); You Ren, Mercer Island, WA (US)

(21) Appl. No.: 19/231,420

(22) Filed: Jun. 6, 2025

Related U.S. Application Data
(60) Provisional application No. 63/657,290, filed on Jun. 7, 2024.

(51) Int. Cl. A63B 24/00

(52) U.S. Cl. CPC A63B 24/0062; A63B 2024/0065

(57) ABSTRACT

Disclosed are methods, systems, and computer-readable media to perform operations including: receiving, by a first machine learning model executed by one or more processors, one or more features related to the workout session; generating, by the first machine learning model and based on the one or more features, a first output including an estimated classification of the user effort for the workout session in a particular category of a plurality of known categories; receiving, by a second machine learning model executed by the one or more processors, the one or more features and the estimated classification output by the first machine learning model; and generating, by the second machine learning model and based on the one or more features and the estimated classification, a second output including an estimated score of the user effort for the workout session.

### CROSS-REFERENCE TO RELATED APPLICATION

This application claims priority to U.S. Provisional Patent Application No. 63/657,290, filed Jun. 7, 2024, the entire content of which is incorporated herein by reference.

### TECHNICAL FIELD

This disclosure relates generally to measuring user effort during a workout session.

### BACKGROUND

A user can wear a fitness device, for example implemented on Apple Watch, iPhone, among others, to track various metrics during a workout session, such as heart rate, elapsed time, average pace, distance covered, calories burned, among others. Fitness equipment such as a treadmill, a cycling bike, among others, can also include a fitness device to track the above-mentioned parameters.

### SUMMARY

According to one innovative aspect of the present disclosure, a method for estimating a user effort for a workout session is disclosed. In one aspect, the method can include receiving, by a first machine learning model executed by one or more processors, one or more features related to the workout session; generating, by the first machine learning model and based on the one or more features, a first output including an estimated classification of the user effort for the workout session in a particular category of a plurality of known categories; receiving, by a second machine learning model executed by the one or more processors, the one or more features and the estimated classification output by the first machine learning model; and generating, by the second machine learning model and based on the one or more features and the estimated classification, a second output including an estimated score of the user effort for the workout session.

Other aspects include apparatuses, systems, and computer programs for performing the aforementioned method.

The innovative method can include other optional features. For example, in some implementations, the method can further include adjusting the estimated score based on prior user effort scores.

In some implementations, the first machine learning model includes a classifier configured to estimate the particular category of the user effort among the plurality of known categories.

In some implementations, the second machine learning model includes a regressor configured to generate the estimated score associated with the particular category.

In some implementations, the classifier is an extreme Gradient Boost (XGBoost) classifier, and the regressor is an XGBoost regressor.

In some implementations, the estimated classification of the user effort is based on an intensity of the workout session, a duration of the workout session, or a combination thereof.

In some implementations, the intensity of the workout session is determined based on one or more of a heart rate with respect to an anaerobic threshold (AT), oxygen consumption with respect to the AT, a degree of depletion of an anaerobic capacity reserve, or changes in intensity over a period of time.

In some implementations, the estimated score is a numeric score within a known range, where different subsets of the known range correspond to different categories of the plurality of known categories.

In some implementations, the features include one or more of maximal oxygen consumption (VO2Max), a maximal heart rate (HRMax), a workout type, a workout duration, a heart rate, an elevation, a speed, changes in intensity over a period of time, an anaerobic threshold (AT), environmental factors, or a Global Positioning System (GPS) signal.

In some implementations, the method can further include performing a validity check to determine whether the workout session is eligible for estimating a score of the user effort.

In some implementations, performing the validity check includes determining whether heart rate data is collected for at least a threshold percentage of time during the workout session.

In some implementations, performing the validity check includes determining whether a duration of the workout session is greater than or equal to a minimum threshold duration.

In some implementations, the method can further include presenting, on a user interface, one or more of the estimated score.

In some implementations, the method can further include generating a graph including estimated scores; and presenting the graph on the user interface.

In some implementations, the method can further include determining a particular type of workout performed for the workout session, the particular type being one of a plurality of known workout types; and in response to determining the particular type of workout, selecting the first machine learning model from a plurality of candidate machine learning models, the first machine learning model configured to generate the estimated classification corresponding to the particular type of workout.

In some implementations, the method can further include determining a particular type of workout performed for the workout session, the particular type being one of a plurality of known workout types; and in response to determining the particular type of workout, selecting the second machine learning model from a plurality of candidate machine learning models, the second machine learning model configured to generate the estimated score corresponding to the particular type of workout.

According to another innovative aspect of the present disclosure, a fitness device for estimating a user effort for a workout session is disclosed. In one aspect, the fitness device can include at least one processor; and a memory storing instructions thereon that, when executed by the at least one processor, cause the at least one processor to perform operations including: receiving, by a first machine learning model, one or more features related to a workout session; generating, by the first machine learning model based on the one or more features, a first output including an estimated classification of user effort for the workout session in a particular category of a plurality of known categories; receiving, by a second machine learning model, the one or more features and the estimated classification output by the first machine learning model; and generating, by the second machine learning model and based on the one or more features and the estimated classification, a second output including an estimated score of the user effort for the workout session.

The innovative fitness device can include other optional features. For example, in some implementations, the estimated classification of the user effort is based on an intensity of the workout session, a duration of the workout session, or a combination thereof.

According to another innovative aspect of the present disclosure, a non-transitory, computer-readable storage medium for estimating a user effort for a workout session is disclosed. In one aspect, a non-transitory, computer-readable storage medium having instructions stored thereon, that when executed by at least one processor, cause the at least one processor to perform operations including: receiving, by a first machine learning model, one or more features related to a workout session; generating, by the first machine learning model based on the one or more features, a first output including an estimated classification of user effort for the workout session in a particular category of a plurality of known categories; receiving, by a second machine learning model, the one or more features and the estimated classification output by the first machine learning model; and generating, by the second machine learning model and based on the one or more features and the estimated classification, a second output including an estimated score of the user effort for the workout session.

The innovative method can include other optional features. For example, in some implementations, the estimated score is a numeric score within a known range, where different subsets of the known range correspond to different categories of the plurality of known categories.

Particular implementations disclosed herein provide one or more of the following advantages. The implementations herein can generate a user effort score for a workout session, so that a user can have an intuitive understanding of how much effort is put in the workout session. The implementations herein can also generate a graph showing user effort scores for multiple workout sessions in a period of time (for example, one month), so that the user can have an intuitive understanding of a workout intensity trend in the period of time.

The details of one or more implementations of the subject matter are set forth in the accompanying drawings and the description below. Other features, aspects, and advantages of the subject matter will become apparent from the description, the drawings, and the claims.

### BRIEF DESCRIPTION OF THE DRAWINGS

FIG. 1 illustrates an example user interface (UI) of a fitness device, according to some implementations.

FIG. 2 illustrates a block diagram of a user effort estimation device for estimating user effort in a workout session, according to some implementations.

FIG. 3 illustrates example features, according to some implementations.

FIG. 4 illustrates an example model mapping workout intensity and duration to user effort categories and scores, according to some implementations.

FIG. 5 illustrates a block diagram of an example bias corrector, according to some implementations.

FIG. 6 illustrates an example process for estimating a user effort score for a workout session, according to some implementations.

FIG. 7 is a schematic illustration of an example computing device that enables the user effort estimation device to predict user effort in a workout session, according to some implementations.

### DETAILED DESCRIPTION

This disclosure is directed to generating a user effort score for a workout session. Features related to a workout session (for example, walk, run, cycling) are input into a classifier, for example, an extreme gradient boosting (XGBoost or XGB) classifier, to obtain an initial classification or a category of workout effort. The various features include workout type, duration, heart rate, elevation, speed, Global Positioning System (GPS) signal, environmental factors (for example, weather, humidity, altitude, among others), user metrics such as age, height, weight, body mass index (BMI), maximal oxygen consumption (VO₂Max), among others. The initial classification or category is one of several known categories (for example, “all-out,” “hard,” “moderate,” or “easy”). The features and the initial classification are input to a regressor (for example, an XGBoost or XGB regressor) to generate an effort score (1-10) for this workout session. The effort score can then be input into a bias corrector to adjust the effort score based on user history data (prior user effort scores). The output of the bias corrector is an adjusted effort score. The adjusted effort score can be displayed on a user interface (UI), for example, a UI of a fitness device, such as a fitness tracker (for example an Apple Watch among others), a mobile phone (for example iPhone among others) associated with the user, a wearable device (for example Apple Vision Pro, a virtual reality headset, an augmented reality headset, a mixed reality headset, among others), or a workout device (for example, a treadmill, a cycling bike, among others).

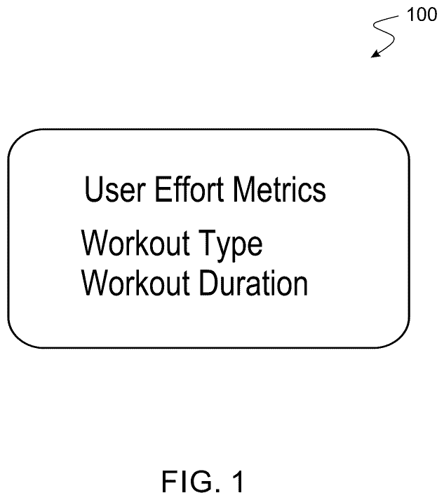

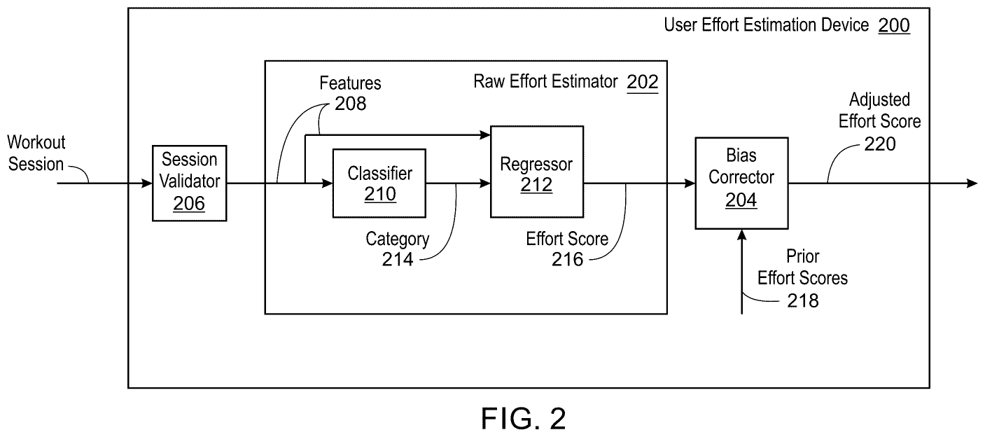

FIG. 1 illustrates an example UI 100 of a fitness device, according to some implementations. The UI 100 includes “user effort metrics” such as a user effort score and/or a user effort category, a “workout type” such as walking, running, among others, and “workout duration”. As an example, a user performed an outdoor run (“workout type”) for 30 minutes (“workout duration”) wearing a fitness device. A user effort category “moderate” (“user effort metrics”) and a user effort score “6” (“user effort metrics”) indicating the workout effort that the user put into the outdoor run, can be shown on a UI 100 provided on a display of the fitness device, following one or more computations (for example performed by raw effort estimator 202 of FIG. 2) that are performed as described in the following sections.

FIG. 2 illustrates a block diagram of a user effort estimation device 200 for estimating user effort in a workout session, according to some implementations. The user effort estimation device 200 includes a raw effort estimator 202, a bias corrector 204, and a session validator 206. The raw effort estimator 202 includes a classifier 210 and a regressor 212. In some implementations, the user effort estimation device 200 is similar to device. In some implementations, the user effort estimation device 200 is implemented using a different computing device, for example, another wearable device, a smartphone, a laptop, a desktop, or a tablet, among other suitable devices. In some implementations, the user effort estimation device 200 is implemented using one or more network connected servers.

In some implementations, a workout session refers to a period of indoor or outdoor physical exercise. When a user engages in a workout session, the session validator 206 checks the validity of the workout session, for example, to determine whether the workout session is eligible for user effort estimation. In some examples, a workout session is eligible for user effort estimation when the duration of the workout session is equal to or greater than a threshold value for user effort estimation. The threshold value can be, for example, five or ten minutes, or some other suitable time duration. In some examples, the heart rate (HR) data is acquired (for example, at least every five seconds) for at least 50% of the duration of the workout session. If the validity check is satisfied, the session validator 206 obtains one or more features 208 related to the workout session, and provides these features 208 as inputs to the classifier 210 and regressor 212 in the raw effort estimator 202.

In some implementations, the classifier 210 and the regressor 212 are two machine learning models. A classifier 210 is used for classification tasks to predict a category or classification of an input. The output of the classifier 210 is discrete and usually categorical. Examples of classifier 210 include decision trees, support vector machines, and neural networks. In some implementations, the classifier 210 is an XGB classifier. A regressor 212 is used for regression tasks to predict a continuous value. Unlike the classifier 210, the output of a regressor 212 is a continuous number. Examples of a regressor 212 include linear regression, polynomial regression, and neural networks. In some implementations, the regressor 212 is an XGB regressor.

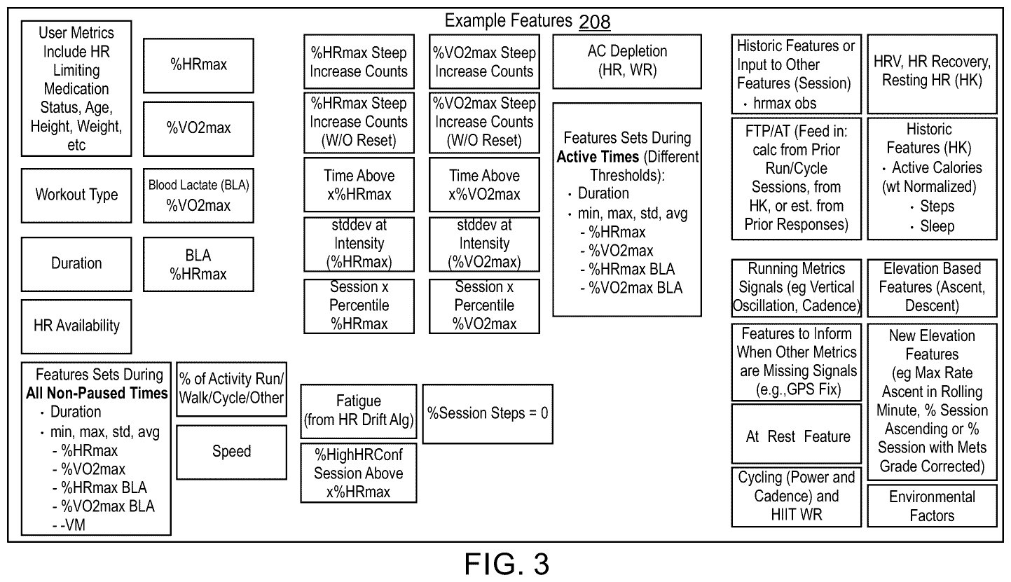

Features 208 include workout type, workout duration, heart rate, elevation, speed, GPS signal, user metrics such as age, height, weight, BMI, VO₂Max, among others. FIG. 3 illustrates a list of example features 208, according to some implementations. Heart rate (HR) and work rate (WR, indicated by oxygen consumption) associated with blood lactate (BLA) represent workout intensity. In some implementations, classifier 210 can identify a user's aerobic and anaerobic thresholds. The aerobic threshold is the exercise intensity level at which the user's body transitions from primarily using aerobic metabolism (with oxygen) to start to use anaerobic metabolism (without oxygen) as an additional energy source. It represents the highest workload that can be sustained aerobically before lactic acid (blood lactate) starts increasing in the muscles and the bloodstream. At intensities below the aerobic threshold, the human body can clear lactate from the blood, allowing for sustained energy production with minimal fatigue. The anaerobic threshold (AT) is a higher level of exercise intensity compared to the aerobic threshold. It marks the point at which the body starts to predominantly rely on anaerobic metabolism to generate additional energy, leading to a rapid increase in blood lactate levels in the blood. Beyond the anaerobic threshold, the human body cannot remove lactate from the bloodstream as quickly as it is produced. This accumulation leads to an increased rate of fatigue and a decrease in performance over time. The anaerobic threshold is often reached during high-intensity activities that can only be sustained for a relatively short period. In some implementations, the classifier 210 estimates the initial category 214, from one of several different known categories, of the user effort during the workout session based on the aerobic threshold and the anaerobic threshold. As an example, when the heart rate or oxygen consumption is below about 60% of the maximum heart rate (HRMax) or VO₂Max, respectively, of the user, the classifier 210 can determine that the workout intensity is below the aerobic threshold, and estimate the initial category 214 as “easy.” When the heart rate or oxygen consumption is above about 80% of HRMax or VO₂Max, respectively, of the user, the classifier 210 can determine that the workout intensity is above the anaerobic threshold, and estimate the initial category 214 as “hard” or “all-out.” When the heart rate or oxygen consumption is between about 60% of HRMax or VO₂Max and about 80% of HRMax or VO₂Max, respectively, of the user, the classifier 210 can determine that the workout intensity is between the aerobic threshold and the anaerobic threshold, and estimate the initial category 214 as “Moderate.” The anaerobic threshold (AT) can be predetermined for example, as an average AT of a population (for example, two hundred runners).

In some implementations, the BLA-based features BLA % HRMax or BLA % VO₂Max can translate normalized HR and WR intensity to blood lactate values using an exponential relationship. The exponential curve can be population-based (a common exponential curve for every user) or personalized with an estimated anaerobic threshold (each user has a personalized exponential curve).

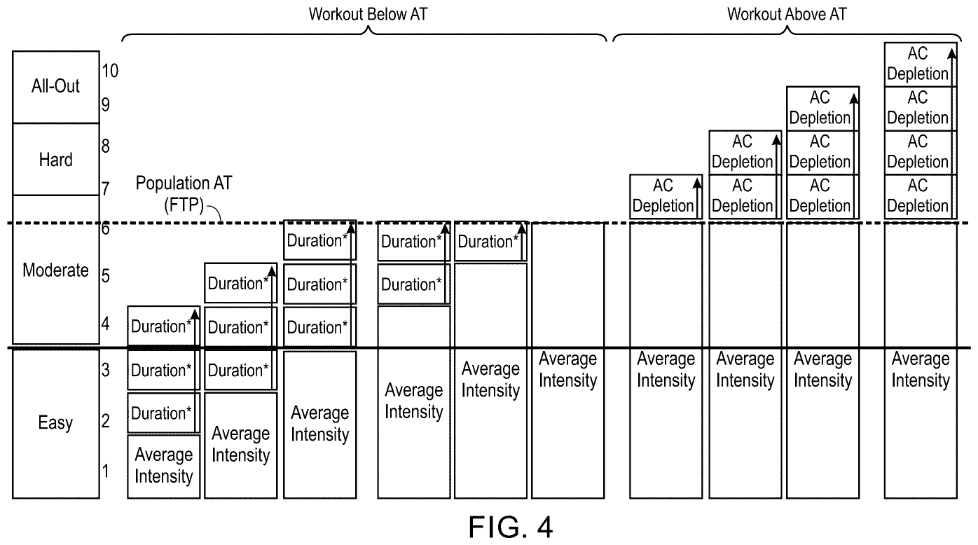

The features based on Anaerobic Capacity (e.g., the amount of Anaerobic Capacity Depletion and an effort score generated in a model of FIG. 4) can use population-based AT (a common AT for every user) or be personalized with an estimated anaerobic threshold (each user has his/her own AT). Personalized AT is associated with an intensity threshold when depletion of anaerobic capacity reserves begins, the amount of anaerobic capacity reserves, and an increase in an effort score as anaerobic capacity reserves are depleted.

In some examples, the session validator 206 provides changes in intensity as a feature input to the classifier 210 to be used to estimate the initial category 214. For example, the classifier 210 can calculate a standard deviation (stddev) of heart rate samples or oxygen consumption samples that are more than about 80% of HRMax or VO₂Max (corresponding to the anaerobic threshold) to indicate variability in intensity. In some cases, the classifier 210 can detect a steep increase in intensity using a sliding window (a period of time). The sliding window can be, for example, 15 seconds, 30 seconds, 45 seconds, 60 seconds, among others. The classifier 210 can determine heart rate samples or oxygen consumption samples having an increase of 8% ˜10% of HRMax or VO2Max with respect to 50% of HRMax or VO₂Max as a steep increase within a sliding window. For steeper increases, the possibility of the initial category 214 being “hard” or “all-out” is higher.

In some examples, the session validator 206 provides workout duration as a feature input to the classifier 210 to be used to estimate the initial category. As an example, the longer the duration during which the heart rate or oxygen consumption is more than about 80% of HRMax or VO₂Max (corresponding to the anaerobic threshold), the higher the possibility that the classifier 210 determines the initial category 214 to be “hard” or “all-out.”

The classifier 210 provides the estimated category 214 as an input to the regressor 212. This is in addition to the features 208 that are also provided as inputs to the regressor 212 by the session validator 206.

FIG. 4 illustrates an example model mapping workout intensity and duration to user effort categories and scores, according to some implementations. As shown, when the workout intensity is lower than the AT or depletion of anaerobic capacity reserves (AC depletion) is less than a predetermined threshold (for example, 10%), the model determines the initial category 214 (“easy” or “moderate”) and the user effort score 216 (for example, 1-6), respectively, based on average intensity and duration of the workout session. When the workout intensity is higher than the AT or the AC depletion is more than the predetermined threshold (for example, 10%), the model determines the initial category 214 (top of “moderate,” “hard,” or “all-out”) and the user effort score 216 (for example, 6-10), respectively, based on the AC depletion. In some implementations, a functional threshold power (FTP) can be applied to determine the initial category 214 and the user effort score 216, in addition to or as an alternative to the AT. In this context, FTP is the highest power output a user can sustain for one hour. FTP can be a metric for cycling users, for example, and is a practical measure of a user's endurance and aerobic capacity. The model can combine features such as average intensity, duration, and AC depletion over the course of the workout session to generate an effort score. In some implementations, the effort score generated by the model can be an additional feature input to the classifier 210 and the regressor 212. In some implementations, the effort score generated by the model can be provided to a user (e.g., displayed on UI 100 of FIG. 1).

Referring back to FIG. 2, the classifier 210 can determine (that is, estimate or predict) an initial category 214 of user effort in the workout session, such as “all-out,” “hard,” “moderate,” or “easy.” The initial category 214 and one or more features 208 are then input into the regressor 212, which fine-tunes the initial category 214 based on the provided features 208 to determine the user effort category with greater precision, and outputs a corresponding user effort score 216 in a range of 1-10.

In some implementations, the user effort score 216 output by the regressor 212 is input to bias corrector 204. Based on user history data, including, for example, prior effort scores 218 for prior workout sessions. The bias corrector 204 adjusts the effort score and outputs adjusted user effort score 220. As an example, the bias corrector 204 may have recorded that in the most recent three months, a user often changed user effort scores 216 for prior workout sessions upward (for example, “6” to “7” or “7” to “8”, among others). Learned from the user's prior behavior of changing the user effort score upward, the bias corrector 204 can adjust the current user effort score 216 to be one higher, for example, increasing a current effort score 216 (for example, “5”) by +1 to output an adjusted effort score 220 (for example, “6”). As another example, a user may have completed an intense first workout session 30 minutes ago, and then initiated a second workout session. The bias corrector 204 can adjust the user effort score 216 of the second workout session to be one higher (for example, +1), taking into account the user's fatigue due to the first workout session 30 minutes ago.

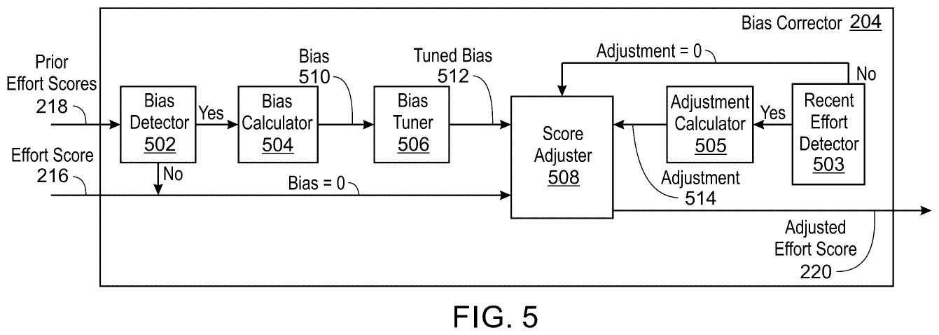

FIG. 5 illustrates a block diagram of an example bias corrector 204, according to some implementations. The bias corrector 204 includes bias detector 502, bias calculator 504, bias tuner 506, recent effort detector 503, adjustment calculator 505, and score adjuster 508. The bias detector 502 determines whether a bias is present in prior user effort scores 218 of prior workout sessions (for example, workout sessions within the previous two or three months). If a user changed prior effort scores predicted by the user effort estimation device 200, downward (for example, “6” to “5” or “8” to “7”, among others) frequently (for example, changed prior effort scores downward for at least three workout sessions), the bias detector 502 determines that a bias 510 is present in prior user effort scores 218; otherwise bias=0. The bias calculator 504 calculates the bias 510 to be a function of the prior predicted score. The function can be, for example, mean(prior predicted score−prior final score). The prior predicted score refers to a prior effort score 216 output by the raw effort estimator 202. The prior final score refers to a final score determined by adjusting the prior effort score 216 (for example adjusted by a user).

The bias tuner 506 tunes or caps the bias 510 to generate a tuned bias 512. For example, the bias tuner 506 caps the bias 510 in a range of [−2, 2]. The tuned bias 512 is input into the score adjuster 508. The recent effort detector 503 determines whether the user completed one or more intense workout sessions (for example, corresponding to final user effort score more than 6) within a previous time period (for example, 4 hours) from the current workout session. If one or more recent intense workout sessions are present within the previous time period, the adjustment calculator 505 calculates an adjustment value 514. If no intense workout sessions are present within the predetermined period of time, the adjustment value is set to 0. The adjustment calculator 505 can calculate the adjustment value 514 based on an effort score of each recent intense workout session and a time interval between the current workout session and each recent intense workout session. In some examples, the adjustment value 514 can further be capped in a range of [−2, 2]. The score adjuster 508 calculates and outputs the adjusted effort score 220 as a function of the effort score 216, tuned bias 512 and adjustment value 514. For example, the adjusted effort score 220 can be effort score 216−(tuned bias 512−adjustment value 514). In some implementations, the adjusted user effort score 220 is provided to the user, for example, shown on the UI of a fitness device as described with respect to FIG. 1.

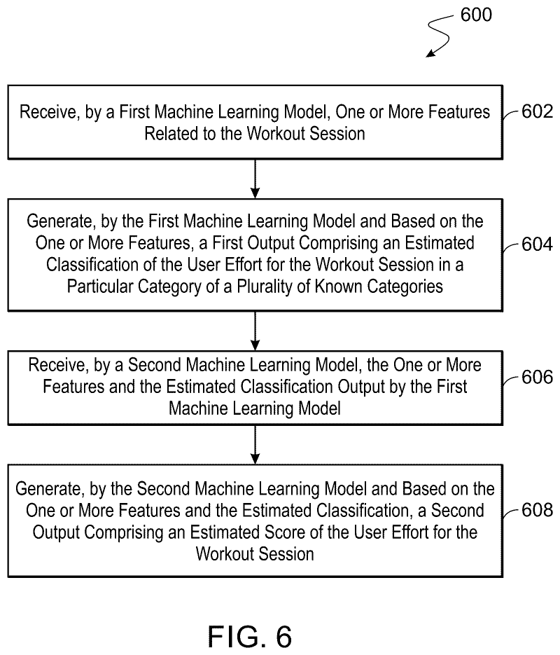

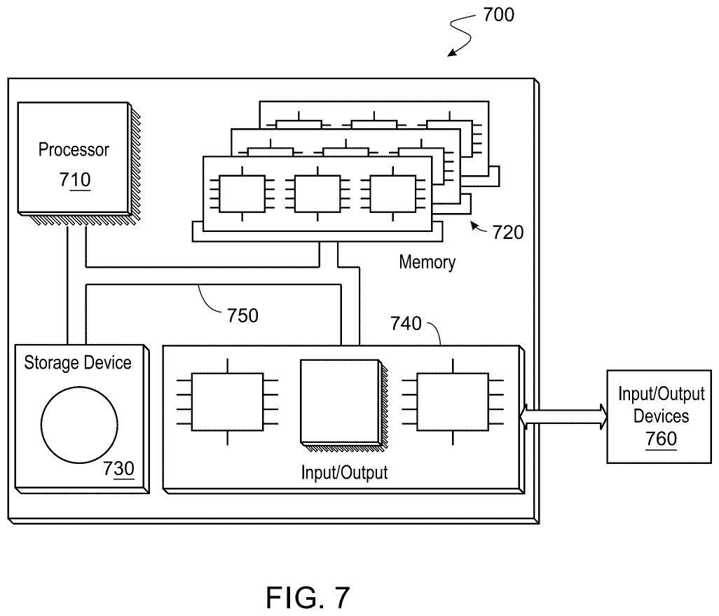

FIG. 6 illustrates an example process for estimating a user effort score for a workout session, according to some implementations. The process 600 is described as being performed by a computing device including one or more processors. For example, in some implementations, the process 600 is performed by user effort estimation device 200. In some implementations, the user effort estimation device 200 is realized using computing device 700 of FIG. 7. The example process 600 shown in FIG. 6 can be modified or reconfigured to include additional, fewer, or different steps (not shown in FIG. 7), which can be performed in the order shown or in a different order.

At 602, a first machine learning model (for example, classifier 210 of FIG. 2) executed by one or more processors (for example, processor 710 of computing device 700 of FIG. 7) receives one or more features related to the workout session. The features include one or more of VO₂Max, HRMax, a workout type, a workout duration, a heart rate, an elevation, a speed, changes in intensity over a period of time, AT, environmental factors, or a GPS signal.

In some implementations, the computing device determines a particular type of workout performed for the workout session. The particular type is one of a plurality of known workout types (for example, jogging, running, cycling, walking, swimming, rowing, rope-jumping, weightlifting, bodyweight exercises such as push-ups, pull-ups, squats, yoga, sports such as soccer, basketball, dancing, among others). In response to determining the particular type of workout, the computing device selects a first machine learning model from a plurality of candidate machine learning models. Different types of workouts may correspond to different first machine learning models. The first machine learning model is configured to generate the estimated classification (for example, category 214 of FIG. 2) corresponding to the particular type of workout.

At 604, the first machine learning model generates, based on the one or more features, a first output including an estimated classification of the user effort for the workout session in a particular category of a plurality of known categories (for example, “all-out,” “hard,” “moderate,” or “easy”).

In some implementations, the first machine learning model includes a classifier configured to estimate the particular category of the user effort among the plurality of known categories (for example, “all-out,” “hard,” “moderate,” or “easy”). In some examples, the classifier is an XGBoost classifier, and the regressor is an XGBoost regressor.

In some implementations, the estimated classification of the user effort can be based on the intensity of the workout session, a duration of the workout session, or a combination thereof. The intensity of the workout session is determined based on one or more of a heart rate with respect to an AT, oxygen consumption with respect to the AT, a degree of depletion of an anaerobic capacity reserve, or changes in intensity over a period of time.

At 606, a second machine learning model (for example, regressor 212 of FIG. 2) executed by one or more processors (for example, processor 710 of computing device 700 of FIG. 7) receives the one or more features and the estimated classification output by the first machine learning model.

In some implementations, the computing device determines a particular type of workout performed for the workout session. The particular type is one of a plurality of known workout types (for example, jogging, running, cycling, walking, swimming, rowing, rope-jumping, weightlifting, bodyweight exercises such as push-ups, pull-ups, squats, yoga, sports such as soccer, basketball, dancing, among others). In response to determining the particular type of workout, the computing device selects the second machine learning model from a plurality of candidate machine learning models. Different types of workouts may correspond to different second machine learning models. The second machine learning model is configured to generate the estimated score (for example, effort score 216 of FIG. 2) corresponding to the particular type of workout.

In some implementations, the second machine learning model includes a regressor configured to generate the estimated score (for example, effort score 216 of FIG. 2) associated with the particular category (for example, category 214 of FIG. 2).

At 608, the second machine learning model generates, based on the one or more features and the estimated classification (for example, category 214 of FIG. 2), a second output including an estimated score (for example, effort score 216 of FIG. 2) of the user effort for the workout session.

In some implementations, the estimated score (for example, effort score 216 of FIG. 2) is a numeric score within a known range (for example, 1-10). Different subsets of the known range (for example, 1-10) correspond to different categories of the plurality of known categories. For example, estimated scores 1-3 correspond to the category “Easy”; estimated scores 4-6 correspond to the category “Moderate”; estimated scores 7-8 correspond to the category “hard”; estimated scores 9-10 correspond to the category “all-out”.

In some implementations, a bias corrector (for example, bias corrector 204) can further adjust the estimated score (for example, effort score 216 of FIG. 2) based on prior user effort scores (for example, prior effort scores 218 of FIG. 2).

In some implementations, a session validator (for example, session validator 206) performs a validity check to determine whether the workout session is eligible for estimating a score of the user effort. In some examples, the validity check includes determining whether heart rate data is collected for at least a threshold percentage (for example, 50%) of time during the workout session. In some examples, the validity check includes determining whether the duration of the workout session is greater than or equal to a minimum threshold duration (for example, five minutes).

In some implementations, the computing device presents, on a UI 100, an estimated score. In some examples, the computing device generates a graph including estimated scores in multiple workout sessions and presents the graph on the UI 100 of a fitness device.

FIG. 7 is a schematic illustration of an example computing device 700 (or control system). In some implementations, the computing device 700 implements user effort estimation device 200 to predict user effort in a workout session, according to some implementations. The computing device 700 can be integrated into a fitness device, a mobile phone carried by a user during the workout session, or fitness equipment (a treadmill, a cycling bike, among others). For example, the computing device 700 may be operable according to the process 600 of FIG. 6. The computing device 700 is intended to include various forms of digital computers, such as printed circuit boards (PCB), processors, digital circuitry, field programmable gate arrays (FPGAs), among others.

The computing device 700 includes a processor 710, a memory 720, a storage device 730, and an input/output interface 740 communicatively coupled with input/output devices 760 (for example, a UI screen and sensors such as a heart rate sensor, an accelerometer, SpO2 Sensor, a GPS/GNSS sensor, among others). Each of the components 710, 720, 730, and 740 are interconnected using a system bus 750. The processor 710 is capable of processing instructions for execution within the computing device 700. The processor may be designed using any of a number of architectures. For example, the processor 710 may be a Complex Instruction Set Computers (CISC) processor, a Reduced Instruction Set Computer (RISC) processor, or a Minimal Instruction Set Computer (MISC) processor.

In one implementation, the processor 710 is a single-threaded processor. In another implementation, the processor 710 is a multi-threaded processor. The processor 710 is capable of processing instructions stored in the memory 720 or on the storage device 730 to display graphical information for a user interface on the input/output interface 740.

The memory 720 stores information within the computing device 700. In one implementation, the memory 720 is a computer-readable medium. In one implementation, the memory 720 is a volatile memory unit. In another implementation, the memory 720 is a non-volatile memory unit.

The storage device 730 is capable of providing mass storage for the computing device 700. In one implementation, the storage device 730 is a computer-readable medium. The storage device 730 can be used to store sensor data, user effort scores, among others.

The input/output interface 740 provides input/output operations for the computing device 700. In some implementations, the input/output devices 760 include a display unit for displaying graphical user interfaces. In some implementations, the input/output devices 760 include sensors for tracking features of a workout session, such as a heart rate sensor, an accelerometer, a SpO2 sensor, a GPS/GNSS sensor, among others.

In the foregoing description, aspects and embodiments of the present disclosure have been described with reference to numerous specific details that can vary from implementation to implementation. Accordingly, the description and drawings are to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction. Any definitions expressly set forth herein for terms contained in such claims shall govern the meaning of such terms as used in the claims. In addition, when we use the term “further comprising” or “further including” in the foregoing description or following claims, what follows this phrase can be an additional step or entity, or a sub-step/sub-entity of a previously-recited step or entity.

We claim:

1. A method for estimating a user effort for a workout session, the method comprising:
receiving, by a first machine learning model executed by one or more processors, one or more features related to the workout session;
generating, by the first machine learning model and based on the one or more features, a first output comprising an estimated classification of the user effort for the workout session in a particular category of a plurality of known categories;
receiving, by a second machine learning model executed by the one or more processors, the one or more features and the estimated classification output by the first machine learning model; and
generating, by the second machine learning model and based on the one or more features and the estimated classification, a second output comprising an estimated score of the user effort for the workout session.

2. The method of claim 1, further comprising:
adjusting the estimated score based on prior user effort scores.

3. The method of claim 1, wherein the first machine learning model comprises a classifier configured to estimate the particular category of the user effort among the plurality of known categories.

4. The method of claim 3, wherein the second machine learning model comprises a regressor configured to generate the estimated score associated with the particular category.

5. The method of claim 4, wherein the classifier is an extreme Gradient Boost (XGBoost) classifier, and the regressor is an XGBoost regressor.

6. The method of claim 1, wherein the estimated classification of the user effort is based on an intensity of the workout session, a duration of the workout session, or a combination thereof.

7. The method of claim 6, wherein the intensity of the workout session is determined based on one or more of a heart rate with respect to an anaerobic threshold (AT), oxygen consumption with respect to the AT, a degree of depletion of an anaerobic capacity reserve, or changes in intensity over a period of time.

8. The method of claim 1, wherein the estimated score is a numeric score within a known range, where different subsets of the known range correspond to different categories of the plurality of known categories.

9. The method of claim 1, wherein the features comprise one or more of maximal oxygen consumption (VO₂Max), a maximal heart rate (HRMax), a workout type, a workout duration, a heart rate, an elevation, a speed, changes in intensity over a period of time, an anaerobic threshold (AT), environmental factors, or a Global Positioning System (GPS) signal.

10. The method of claim 1, further comprising:
performing a validity check to determine whether the workout session is eligible for estimating a score of the user effort.

11. The method of claim 10, wherein performing the validity check comprises determining whether heart rate data is collected for at least a threshold percentage of time during the workout session.

12. The method of claim 10, wherein performing the validity check comprises determining whether a duration of the workout session is greater than or equal to a minimum threshold duration.

13. The method of claim 1, further comprising:
presenting, on a user interface, the estimated score.

14. The method of claim 13, further comprising:
generating a graph comprising estimated scores in different workout sessions; and
presenting the graph on the user interface.

15. The method of claim 1, further comprising:
determining a particular type of workout performed for the workout session, the particular type being one of a plurality of known workout types; and
in response to determining the particular type of workout, selecting the first machine learning model from a plurality of candidate machine learning models, the first machine learning model configured to generate the estimated classification corresponding to the particular type of workout.

16. The method of claim 1, further comprising:
determining a particular type of workout performed for the workout session, the particular type being one of a plurality of known workout types; and
in response to determining the particular type of workout, selecting the second machine learning model from a plurality of candidate machine learning models, the second machine learning model configured to generate the estimated score corresponding to the particular type of workout.

17. A fitness device, comprising:
at least one processor; and
a memory storing instructions thereon that, when executed by the at least one processor, cause the at least one processor to perform operations comprising:
receiving, by a first machine learning model, one or more features related to a workout session;
generating, by the first machine learning model based on the one or more features, a first output comprising an estimated classification of user effort for the workout session in a particular category of a plurality of known categories;
receiving, by a second machine learning model, the one or more features and the estimated classification output by the first machine learning model; and
generating, by the second machine learning model and based on the one or more features and the estimated classification, a second output comprising an estimated score of the user effort for the workout session.

18. The fitness device of claim 17, wherein the estimated classification of the user effort is based on an intensity of the workout session, a duration of the workout session, or a combination thereof.

19. A non-transitory, computer-readable storage medium having instructions stored thereon, that when executed by at least one processor, cause the at least one processor to perform operations comprising:
receiving, by a first machine learning model, one or more features related to a workout session;
generating, by the first machine learning model based on the one or more features, a first output comprising an estimated classification of user effort for the workout session in a particular category of a plurality of known categories;
receiving, by a second machine learning model, the one or more features and the estimated classification output by the first machine learning model; and
generating, by the second machine learning model and based on the one or more features and the estimated classification, a second output comprising an estimated score of the user effort for the workout session.

20. The computer-readable storage medium of claim 19, wherein the estimated score is a numeric score within a known range, where different subsets of the known range correspond to different categories of the plurality of known categories.
